(19) United States
(12) Patent Application Publication
(10) Pub. No.: US 2013/0174863 A1
(43) Pub. Date: Jul. 11, 2013

(54) HAIR CARE COMPOSITIONS

(71) Applicant: THE PROCTER & GAMBLE COMPANY, Cincinnati, OH (US)

(72) Inventors: Jennifer Mary Marsh, Mason, OH (US); Howard David Hutton, III, Oregonia, OH (US); Kevin Lee Doyle, Fairfield, OH (US); Jennifer Elaine Hilvert, Cincinnati, OH (US)

(73) Assignee: THE PROCTER & GAMBLE COMPANY, Cincinnati, OH (US)

(21) Appl. No.: 13/737,035

(22) Filed: Jan. 9, 2013

Related U.S. Application Data
(60) Provisional application No. 61/584,515, filed on Jan. 9, 2012.

(51) Int. Cl. A61K 8/44; A61Q 5/02; A61K 8/02

(52) U.S. Cl. CPC A61K 8/44; A61K 8/0216; A61Q 5/02
USPC 132/202; 510/119; 510/120

(57) ABSTRACT

A hair care composition that inhibits redox metal deposit build up on keratinous tissue is provided, the composition includes a chelant, a low pH buffer system, a detersive surfactant, and a carrier.

### FIELD OF THE INVENTION

The present composition relates to a low pH hair care composition containing a buffer system, and a chelant, wherein the composition inhibits mineral deposit build up on hair, and methods of using the same.

### BACKGROUND OF THE INVENTION

Many consumers use water supplies that originate from ground water sources, which often contain various quantities of dissolved minerals such as calcite (calcium), dolomite (calcium and magnesium), magnetite (iron), and chalcanthite (copper). It has been found that even trace quantities of these minerals can deposit on the hair surface and in between the cuticle layers of hair. This deposition of minerals on hair is especially problematic for the consumers living in areas where their water source is hard, e.g., contains elevated concentrations of minerals such as calcium, magnesium, copper, and iron salts, and can lead to poor hair health. For example, deposits of calcium salts, such as calcium carbonate, between the cuticle layers can lead to poor shine and accelerate cuticle damage. Further, deposits of transition metal ions, such as copper and iron, can facilitate reduction-oxidation (redox) reactions during hair coloring treatments and generate hydroxyl radicals, which in turn can cause damage to the hair, as well as reduced color uptake. Accordingly, there is a need for hair care compositions that can inhibit minerals depositing on keratinous tissue, as well as facilitate the removal of minerals already deposited thereon.

### SUMMARY OF THE INVENTION

Embodiments of the present invention are directed to a hair care compositions, and methods of using the same, useful for inhibiting or removing mineral deposit build up on hair.

According to an embodiment, a hair care composition is provided, comprising from about 0.01 wt % to about 10 wt % of ethylene diamine disuccinic acid (EDDS) or salts thereof; a buffer system comprising an organic acid and/or a salt thereof, wherein the organic acid is selected from an alpha-hydroxy acid or a polycarboxylic acid; from about 2 wt % to about 50 wt % of a detersive surfactant; and a carrier. The buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).

According to another embodiment, a hair care composition is provided, comprising from about 0.01 wt % to about 10 wt % of a chelant (L), a buffer system comprising an organic acid and/or a salt thereof, wherein the organic acid is selected from an alpha-hydroxy acid or a polycarboxylic acid; from about 2 wt % to about 50 wt % of a detersive surfactant; and a carrier. The chelant has a log K_{CaL} of less than about −2, and a log K_{CuL} of greater than about 3 or a log K_{FeL} of greater than about 10. The log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5. The buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).

According to another embodiment, a method of reducing deposited mineral content on keratinous tissue is provided, comprising contacting keratinous tissue with a hair care composition; and rinsing the hair care composition from the keratinous tissue. The hair care composition comprises from about 0.01 wt % to about 10 wt % of a chelant (L), wherein the chelant has a log K_{CaL} of less than about −2, and a log K_{CuL} of greater than about 3 or a log K_{FeL} of greater than about 10; a buffer system comprising an organic acid and/or a salt thereof, wherein the organic acid is selected from an alpha-hydroxy acid or a polycarboxylic acid; from about 2 wt % to about 50 wt % of a detersive surfactant; and a carrier. The buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v);

According to another embodiment, a hair care composition in the form of a porous dissolvable solid structure is provided, comprising from about 0.01 wt % to about 10 wt % of a chelant (L), wherein the chelant has a log K_{CaL} of less than about −2, and a log K_{CuL} of greater than about 3 or a log K_{FeL} of greater than about 10; a buffer system comprising an organic acid and/or a salt thereof, wherein the organic acid is selected from an alpha-hydroxy acid or a polycarboxylic acid, wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v); from about 23 wt % to about 75 wt % of a detersive surfactant; from about 10 wt % to about 50 wt % of a water soluble polymer; and optionally, from about 1 wt % to about 15 wt % of a plasticizer; such that the hair care composition is in the form of a porous dissolvable solid structure, wherein said structure has a Percent open cell content of from about 80 wt % to about 100 wt %.

According to another embodiment, a hair care composition in the form of a porous dissolvable solid structure is provided, comprising from about 0.01 wt % to about 10 wt % of a chelant (L), wherein the chelant has a log K_{CaL} of less than about −2, and a log K_{CuL} of greater than about 3 or a log K_{FeL} of greater than about 10; a buffer system comprising an organic acid and/or a salt thereof, wherein the organic acid is selected from an alpha-hydroxy acid or a polycarboxylic acid, wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v); from about 23 wt % to about 75 wt % of a detersive surfactant; wherein said detersive surfactant has an average ethoxylate/alkyl ratio of from about 0.001 to about 0.45; from about 10 wt % to about 50 wt % of a water soluble polymer; and from about 1 wt % to about 15 wt % of a plasticizer; and wherein said structure has a density of from about 0.03 g/cm³ to about 0.20 g/cm³.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

### DEFINITIONS

As used herein, the term “hair care composition” refers to the combination of a chelant, a buffer system, a surfactant, and carrier.

As used herein, the term “low pH” refers to a pH range from about 2 to about 6 at 25° C.

As used herein, the term “fluid” includes liquids and gels.

As used herein, the term “log x” refers to the common (or decadic) logarithm of x.

As used herein, the articles including “a” and “an” when used in a claim, are understood to mean one or more of what is claimed or described.

As used herein, the terms “include,” “includes,” and “including,” are meant to be non-limiting and are understood to mean “comprise,” “comprises,” and “comprising,” respectively.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Benefits of Low pH Buffered Chelants

Many water sources that are used by consumers for personal care contain elevated levels of calcium and magnesium salts, as well as undesirable levels of redox metals (e.g., copper and/or iron) salts. As such, using chelants to sequester trace redox metals often proves to be ineffective because most chelants also competitively bind calcium and/or magnesium.

According to one embodiment, it has been found that a chelant having a Conditional Stability Constant, as discussed below, for Calcium (K_{CaL}) below a certain value, in combination with having a Conditional Stability Constant for Copper (K_{CuL}) and/or Iron (K_{FeL}) above a certain level, will demonstrate a sufficient level of selective affinity for these redox metals and thus inhibit the deposition of the same onto hair. Chelants having this selective affinity may also reduce the quantities of redox metals already deposited.

In order to maintain a desired level of the aforementioned selective affinity for the chelant, the pH of the hair care composition further comprises a sufficient quantity of a buffer system comprising an organic acid and/or a salt thereof to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).

Another advantage afforded by the buffer system is the ability of the organic acid and/or the organic acid salt to form a soluble calcium complex, which thereby inhibits the deposition of calcium salts (e.g., calcium carbonate) onto the hair.

Thus, in accordance with an embodiment of the present invention, a hair care composition is provided that comprises from about a chelant, salts and derivatives thereof; a buffer system comprising an organic acid and/or a salt thereof; a detersive surfactant; and a carrier.

### A. Chelants

Chelants are well known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties. This term include alkali metal, alkaline earth, ammonium, substituted ammonium (e.g monoethanolammonium, diethanolammonium, triethanolammonium) salts, esters of chelants having an acidic moiety and mixtures thereof, in particular all sodium, potassium or ammonium salts. The term “derivatives” also includes “chelating surfactant” compounds, such as those exemplified in U.S. Pat. No. 5,284,972, and large molecules comprising one or more chelating groups having the same functional structure as the parent chelants, such as polymeric EDDS (ethylenediaminedisuccinic acid) disclosed in U.S. Pat. No. 5,747,440.

It has been found that chelants possessing a stronger affinity for redox metals (e.g., transition metal ions such as Cu⁺² and/or Fe⁺³) over that of alkaline-earth metal ions such as Ca⁺² at pH about 2 to about 6 efficiently inhibit the deposition of redox metals on keratinous, and can reduce the amount of redox metal salt deposits already existing on the keratinous tissue.

### Conditional Stability Constants of Exemplary Chelants

The relative affinity of a chelant at a specified pH for Cu⁺² versus Ca⁺² can be assessed by comparing the log of the Conditional Stability Constant of the chelant for Cu⁺² to the log of the Conditional Stability Constant of the chelant for Ca⁺² as described below.

The Conditional Stability Constant is a parameter commonly used in the art to practically assess the stability of metal-chelant complex at a given pH. A detailed discussion on Conditional Stability Constant can be found for example in “Dow chelating agents” published by the Dow Chemical Company Limited, incorporated herein by reference. The Conditional Formation Constant for a given metal referred to in this Patent Application is calculated using the following equation:

$${K}_{ML} ( cond ) = \frac{{K}_{ML}}{{α}_{M} \cdot {α}_{HL}} log {K}_{ML} ( cond ) = log {K}_{ML} - log {α}_{HL} - log {α}_{M}$$

wherein K_{ML} is the Stability Constant, α_{HL} is an alpha coefficient of a partially protonated ligand (at a given pH), and α_{MOH} is an alpha coefficient of a metal hydroxide (at a given pH).

The Stability Constant of a metal chelant interaction is defined as:

$${K}_{ML} = \frac{[ ML ]}{[ M ] [ L ]}$$

where:

[ML]=concentration of metal ligand complex at equilibrium

[M]=concentration of free metal ion

[L]=concentration of free ligand in a fully deprotonated form

K_{ML}=stability constant for the metal chelant complex.

All concentrations are expressed in mol/dm³. Stability constants are conveniently expressed as logarithms. The values of the logarithms of the stability constant values for some exemplary metal ion—chelant complexes are given in the following table:

TABLE 1
Log Stability Constants for 1:1 complexes of various chelants with
Cu⁺², Fe⁺³, and Ca⁺² (fully deprotonated chelants) log K* Agent

| | log K* | | |
|---|---|---|---|
| Chelant | Cu | Fe | Ca |
| EDDS | 18.35 | 22.0 | 4.58 |
| DTPMP | 19.5 | 26.5 | 7.1 |
| EDTMP | 23.2 | 6.5 | 9.36 |
| DTPA | 21.4 | 16.4 | 10.75 |
| HEDP | 11.84 | 14.1 | 6.0 |
| EDTA | 18.78 | 14.3 | 10.65 |
| EDDHA (EHPG) | 25.3 | 35.5 | 7.2 |
| HBED | 9.29 | 39.7 | 9.3 |
| EDDG | 15.15 | * | 2.70 |
| HPDDS | 12.84 | * | 2.94 |

*All measured at 25° C. and 0.1M ionic strength
*Not available

Most chelants have a degree of protonation that is dependent on pH. This can be expressed using chelant proton stability constants (stepwise K). These stability constants are obtained from the equation below:

$$H + LHn ⇌ {LH}_{n + 1} {K}_{Hn + 1} = \frac{[ {LH}_{n + 1} ]}{[ H ] [ {LH}_{n} ]}$$

The values of the proton chelant stability constant for a few commonly known chelants are provided in Tables 2a-2d below:

TABLE 2a
Log protonation constants for tetra-dentate chelants [1]

| | HL³⁻ | H₂L²⁻ | H₃L⁻ | H₄L |
|---|---|---|---|---|
| EDDS⁴⁻ | 10.01 | 6.84 | 3.86 | 2.95 |
| HEDP⁴⁻ | 10.8 | 6.88 | 2.53 | 1.8 |
| EDTA⁴⁻ | 10.19 | 6.13 | 2.69 | 2.00 |
| EDDHA⁴⁻ | 12.1 | 9.5 | 8.5 | 6.3 |
| EDDG⁴⁻ | 9.94 | 6.50 | 4.38 | 4.23 |
| HPPDS⁴⁻ | 9.21 | 8.13 | 4.16 | 3.56 |

TABLE 2b
Log protonation constants for penta-dentate chelants [1]

| | HL⁴⁻ | H₂L³⁻ | H₃L²⁻ | H₄L⁻ | H₅L |
|---|---|---|---|---|---|
| DTPA⁵⁻ | 10.48 | 8.60 | 4.28 | 2.6 | 2.0 |

TABLE 2c
Log protonation constants for hepta-dentate chelants [1]

| | HL⁶⁻ | H₂L⁵⁻ | H₃L⁴⁻ | H₄L³⁻ | H₅L²⁻ | H₆L⁻ | H₇L |
|---|---|---|---|---|---|---|---|
| EDTMP⁷⁻ | 13.0 | 9.78 | 7.94 | 6.42 | 5.17 | 3.02 | 1.30 |

TABLE 2d
Log protonation constants for deca-protonated chelants [1]

| | HL⁹⁻ | H₂L⁸⁻ | H₃L⁷⁻ | H₄L⁶⁻ | H₅L⁵⁻ | H₆L⁴⁻ | H₇L³⁻ | H₈L²⁻ | H₉L⁻ | H₁₀L |
|---|---|---|---|---|---|---|---|---|---|---|
| DTPMP¹⁰⁻ | 11.1 | 10.1 | 8.2 | 7.2 | 6.3 | 5.5 | 4.5 | 2.8 | 2.6 | 1.5 |

[1] = Calculated using ACD Labs Version 7.0, pka calculation module (http://www.acdlabs.com/home/).

The stability constants of chelant-metal ion complexes are well documented in the literature for commonly used chelants (see for example=Arthur Martell & Robert M Smith, Critically Selected Stability Constants of Metal Complexes Database, Version 3.0 and above, incorporated herein by reference). When not documented the constants can still be measured using various analytical methods (see “Metal Complexes in Aqueous Solutions”, Martel and Hancock, edition Modem Inorganic Chemistry, p. 226-228, incorporated herein by reference).

The gradual change in ligand species as pH changes can be represented using alpha (α) coefficients, defined as:

$$alpha ( α ) coefficient ( at a given pH ) = \frac{Total concentration of ligand}{Free ligand concentration}$$

For example, in the case of tetra-acid chelants the values can be calculated from:

α_{HL}=1+K₁[H]+K₁K₂[H]²+K₁K₂K₃[H]³+K₁K₂K₃K₄[H]⁴

A further factor affecting metal chelant interactions is the tendency of metals to form hydroxide species as the pH increases. However, as the pH range of the present compositions are acidic (i.e., less than 7), the log alpha value (α_{M}) is considered to be constant and approximately negligible.

By combining stability (K) and alpha (α) constants at a given pH, the formula below provides the effective chelating power of a chelant. This is the Conditional Formation Constant referred to in this Patent Application:

$${K}_{ML} ( cond ) = \frac{{K}_{ML}}{{α}_{M} \cdot {α}_{HL}} log {K}_{ML} ( cond ) = log {K}_{ML} - log {α}_{HL} - log {α}_{M}$$

where, as discussed above, α_{HL} is assumed to be zero for an acid composition.

More discussion on conditional stability constant can be found, for example, in “Dow chelating agents” published by the Dow Chemical Company Limited, incorporated herein by reference. The calculated stability constants for a range of chelants with Fe⁺³, Cu⁺², and Ca⁺² are given below in Table 3:

TABLE 3
Calculated conditional stability constants for chelants with
Fe⁺³, Cu⁺², and Ca⁺² at specified pH.

| | | log Conditional Stability Constant | | |
|---|---|---|---|---|
| | | (@ pH) | | |
| Chelant | pH | Cu⁺² | Ca⁺² | Fe⁺³ |
| EDTA | 2 | 5.45 | −2.65 | * |
| | 3 | 8.29 | 0.19 | * |
| | 4 | 10.46 | 2.36 | * |
| | 5 | 12.45 | 4.35 | * |
| | 6 | 14.24 | 6.14 | * |
| | 7 | 15.55 | 7.45 | * |
| | 8 | 16.60 | 8.50 | * |
| EDDS | 2 | 2.59 | −11.11 | 6.29 |
| | 3 | 6.28 | −7.42 | 9.98 |
| | 4 | 9.20 | −4.50 | 12.90 |
| | 5 | 11.41 | −2.29 | 15.11 |
| | 6 | 13.39 | −0.13 | 17.09 |
| | 7 | 15.06 | 1.36 | 18.76 |
| | 8 | 16.26 | 2.56 | 19.96 |
| GLD | 2 | −0.42 | −7.62 | * |
| | 3 | 3.32 | −3.88 | * |
| | 4 | 5.75 | −1.45 | * |
| | 5 | 7.33 | 0.13 | * |
| | 6 | 8.45 | 1.25 | * |
| | 7 | 9.47 | 2.27 | * |
| | 8 | 10.47 | 3.27 | * |
| DTPA | 2 | 3.34 | −7.26 | * |
| | 3 | 6.88 | −3.72 | * |
| | 4 | 9.85 | −0.75 | * |
| | 5 | 12.24 | 1.64 | * |
| | 6 | 14.31 | 3.71 | * |
| | 7 | 16.31 | 5.71 | * |
| | 8 | 18.22 | 7.62 | * |
| EDDHA | 2 | −3.10 | −21.20 | 7.10 |
| | 3 | 0.90 | −17.20 | 11.10 |
| | 4 | 4.90 | −13.20 | 15.10 |
| | 5 | 8.88 | −9.22 | 19.08 |
| | 6 | 12.72 | −5.38 | 22.92 |
| | 7 | 16.11 | −1.99 | 26.31 |
| | 8 | 19.07 | 0.97 | 29.27 |
| HEDTA | 2 | 3.39 | −5.81 | * |
| | 3 | 6.30 | −2.90 | * |
| | 4 | 8.85 | −0.35 | * |
| | 5 | 10.88 | 1.68 | * |
| | 6 | 12.41 | 3.21 | * |
| | 7 | 13.53 | 4.33 | * |
| | 8 | 14.54 | 5.34 | * |
| DTPMP | 2 | −9.10 | −21.50 | −2.10 |
| | 3 | −5.10 | −17.50 | 1.90 |
| | 4 | −1.10 | −13.50 | 5.90 |
| | 5 | 2.90 | −9.50 | 9.90 |
| | 6 | 6.87 | −5.53 | 13.87 |
| | 7 | 10.68 | −1.72 | 17.68 |
| | 8 | 13.85 | 1.45 | 20.85 |
| MGDA | 2 | 3.80 | −3.10 | * |
| | 3 | 5.84 | −1.06 | * |
| | 4 | 7.08 | 0.18 | * |
| | 5 | 8.11 | 1.21 | * |
| | 6 | 9.11 | 2.21 | * |
| | 7 | 10.11 | 3.21 | * |
| | 8 | 11.11 | 4.21 | * |
| HPPDS | 2 | −4.27 | −14.17 | * |
| | 3 | −0.37 | −10.27 | * |
| | 4 | 2.99 | −6.91 | * |
| | 5 | 5.40 | −4.50 | * |
| | 6 | 7.45 | −2.45 | * |
| | 7 | 9.43 | 0.47 | * |
| | 8 | 11.21 | 1.31 | * |
| HBED | 2 | * | * | 13.54 |
| | 3 | * | * | 17.54 |
| | 4 | * | * | 21.52 |
| | 5 | * | * | 25.39 |
| | 6 | * | * | 28.82 |
| | 7 | * | * | 31.89 |
| | 8 | * | * | 34.85 |

* Not calculated and/or not available

It has been found that levels as low as about 0.01% by weight of chelants having a log K_{CaL}, of less than about −2, and a log K_{CuL} of greater than about 3 or a log K_{FeL} of greater than about 10 at a pH of 5 provide acceptable inhibition of redox metal deposition, as well as an unexpected decrease in the existing redox metal deposits, on hair, wherein the log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5. In one embodiment, a chelant having having a log K_{CaL} of less than about −2, and a log K_{CuL} of greater than about 10 or a log K_{FeL} of greater than about 15 at a pH of 5 provide acceptable inhibition of redox metal deposition, as well as an unexpected decrease in the existing redox metal deposits, on hair. The use of stability constants without taking into account the influence of the pH will give misleading results for the purpose of identifying chelants that will selectively bind to trace redox metals at low levels in hard water, and thereby inhibit the deposition of the same onto hair.

According to yet another embodiment, suitable chelants include those having log K_{CaL}/log K_{CuL} value at a pH of 5 of less 0.3. For example, the value log K_{CaL}/log K_{CuL} value at a pH of 5 may be less than 0.25, 0.20, 0.15, 0.10, 0.05, 0.00, or −0.10. In another embodiment, the chelant has a log K_{CaL}/log K_{CuL} value at a pH of 5 of about −0.2.

Accordingly, in one embodiment, the chelant is selected from diethylenetriamine penta(methylene phosphonic acid) (DTPMP); ethylenediamine-N,N′-diglutaric acid (EDDG); ethylenediamine-N,N′-bis(2-hydroxyphenylacetic acid) (EDDHA); ethylene diamine disuccinic acid (EDDS); glutamic acid diacetic acid (GLDA); hexadentate aminocarboxylate (HBED); 2-hydroxypropylendiamin-N-N′-disucinnic acid (HPDDS); methylglycinediacetic acid (MGDA); salts thereof, derivatives thereof, or mixtures thereof. Accordingly, in one embodiment, the chelant is selected from diethylenetriamine penta(methylene phosphonic acid) (DTPMP); ethylenediamine-N,N′-diglutaric acid (EDDG); ethylenediamine-N,N′-bis(2-hydroxyphenylacetic acid) (EDDHA); ethylene diamine disuccinic acid (EDDS); glutamic acid diacetic acid (GLDA); hexadentate aminocarboxylate (HBED); 2-hydroxypropylendiamin-N-N′-disucinnic acid (HPDDS); methylglycinediacetic acid (MGDA); salts and derivatives thereof, or mixtures thereof. In another embodiment, the chelant is EDDS. The EDDS can be used as one of the enantiomeric forms, S,S-EDDS, R,R-EDDS, R,S-EDDS, or a mixture of these forms. In one embodiment, the EDDS is the S,S-EDDS form, as this form of EDDS has desirable biodegradability.

Levels of such chelants in the hair care compositions can be as low as about 0.01 wt % or even as high as about 10 wt %, but above the higher level (i.e., 10 wt %) significant formulation and/or human safety concerns may arise. In an embodiment, the level of a chelant may be at least about 0.05 wt %, at least about 0.1 wt %, at least about 0.25 wt %, at least about 0.5 wt %, at least about 1 wt %, or at least about 2 wt % by weight of the hair care composition. Levels above about 4 wt % can be used but may not result in additional benefit.

### B. Buffer System

According to embodiments of the invention, the hair care composition has a pH from about 2 to about 6 at 25° C. However, as discussed above, to maintain the selectivity of the chelant for redox metals, the pH may be controlled by a buffer system. Accordingly, from the standpoint of reducing changes in pH during the hair cleaning process, the shampoo composition according to embodiments of the present invention further includes a buffer system to assist in maintaining the pH of the shampoo composition itself from about 2 to about 6, and further maintains the pH of a diluted sample in a range from about 3 to about 6 upon dilution with 10 parts of water to 1 part of hair care composition. This desirable buffer capacity for the hair care composition can be imparted by adding a buffer system, which includes an organic acid and/or a salt thereof and has buffering action in a pH range of from 2 to 6.

In one aspect of the buffering system, the organic acid is selected from an alpha-hydroxy acid, a polycarboxylic acid, or mixtures thereof. Accordingly, the organic acid has an acidic functional group having a pKa of about 4.5 or less. In another embodiment, the organic has a second acidic functional group having a pKa of about 6 or less. The organic acids having multiple acidic functional groups can provide improved buffering capacity relative to their single acidic functional group counterparts. In one aspect, the organic acid may have a molecular weight less than about 500 grams per mole (g/mol) to afford enhanced molar efficiency. For example, the molecular weight of the organic acid may be from about 90 g/mol to about 400 g/mol, from about 100 g/mol to about 300 g/mol, from about 130 g/mol to about 250 g/mol, from about 150 g/mol to about 200, or about 190 g/mole. In another aspect, the organic acid may be soluble in water in an amount greater than about 0.2 moles per liter at 25° C. For example, the water solubility of the organic acid may be about 0.3 mol/L or more, about 0.4 mol/L or more, or about 0.5 mol/L or more.

In one aspect of the buffering system is the ability of the organic acid to form soluble chelates with minerals such as calcium carbonate to thereby reduce the amount of calcium that is available to form insoluble crystals that can deposit on the hair. According to one embodiment, exemplary organic acids possess a log Stability Constant, as discussed above, equal to or greater than about 1.6; equal to or greater than about 1.9; equal to or greater than about 2.0; or equal to or greater than about 2.5.

According to an embodiment, an organic acid having a log Conditional Stability Constant, as discussed above, for calcium (K_{CaL}) at pH 5 equal to or greater than about 1.5 will demonstrate a sufficient level of affinity for calcium and thus inhibit the deposition of the same onto keratinous tissue. According to another embodiment, the organic acid possesses a log Conditional Stability Constant for calcium (K_{CaL}) at pH 5 equal to or greater than 1.7; equal to or greater than 1.8; equal to or greater than 1.9; equal to or greater than 2.0; or about 2.1. According to another embodiment, the organic acids possesses a log K_{CaL} of equal to or greater than about 1.5 at pH of 6. For example, the log K_{CaL} of exemplary organic acids may be about 1.7 or greater; about 1.9 or greater; about 2.0 or greater; or about 2.5 or greater at a pH of 6.

Additionally, the exemplary organic acids may also facilitate removal of the calcium deposits that already exist on the hair. Accordingly, in another embodiment, exemplary organic acids include those acids which form a calcium complex having a water solubility that is greater than the water solubility of calcium carbonate at 25° C.

Additionally, the acidic nature (pH of about 2 to about 6) of the hair care composition helps to solubilize crystals already deposited on the hair. Thus, the hair care composition also is effective toward washing out the existing crystals, which can reduce cuticle distortion and thereby reduce cuticle chipping and damage.

According to embodiments of the invention, the organic acid is selected from an alpha-hydroxy acid, a polycarboxylic acid, or mixtures thereof. In one embodiment, the alpha-hydroxy acid is selected from citric acid, malic acid, tartaric acid, or combinations thereof. In another embodiment, the polycarboxylic acid is malonic acid. In another embodiment, the organic acid is citric acid. Further, examples of the salt of such an organic acid can include its alkali metal salts such as the sodium salt and the potassium salt; its ammonium salt; and its alkanolamine salts such as the triethanolamine salt.

No particular limitation is imposed on the amount of the pH buffering agent to be added, and its amount varies depending on the nature of the compound giving buffering ability. When sodium citrate is used as a primary compound giving the buffering ability, for example, it can be added at a concentration of about 0.5 wt % or higher, 2 wt % or higher, 3 wt % or higher, 4 wt % or higher, or 5 wt % or higher. Accordingly, the hair care composition may comprises about 0.5 wt % to about 8 wt %, about 1 wt % to about 5 wt %; about 1 wt % to about 4 wt %, or about 2 to about 3 wt %, for example, to provide the desired level of buffering capacity.

The hair care compositions of the present invention further include a detersive surfactant and a carrier.

### C. Surfactant

The hair care composition of the present invention includes a detersive surfactant, which provides cleaning performance to the composition. The detersive surfactant in turn comprises an anionic surfactant, amphoteric or zwitterionic surfactants, or mixtures thereof. Various examples and descriptions of detersive surfactants are set forth in U.S. Pat. No. 6,649,155; U.S. Patent Application Publication No. 2008/0317698; and U.S. Patent Application Publication No. 2008/0206355, which are incorporated herein by reference in their entirety.

The concentration of the detersive surfactant component in the hair care composition should be sufficient to provide the desired cleaning and lather performance, and generally ranges from about 2 wt % to about 50 wt %, from about 5 wt % to about 30 wt %, from about 8 wt % to about 25 wt %, or from about 10 wt % to about 20 wt %. Accordingly, the hair care composition may comprise a detersive surfactant in an amount of about 5 wt %, about 10 wt %, about 12 wt %, about 15 wt %, about 17 wt %, about 18 wt %, or about 20 wt %, for example.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the hair care composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is sodium lauryl sulfate or sodium laureth sulfate.

Suitable amphoteric or zwitterionic surfactants for use in the hair care composition herein include those which are known for use in hair care or other personal care cleansing. Concentrations of such amphoteric surfactants range from about 0.5 wt % to about 20 wt %, and from about 1 wt % to about 10 wt %. Non limiting examples of suitable zwitterionic or amphoteric surfactants are described in U.S. Pat. Nos. 5,104,646 and 5,106,609, which are incorporated herein by reference in their entirety.

Amphoteric detersive surfactants suitable for use in the hair care composition include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Exemplary amphoteric detersive surfactants for use in the present hair care composition include cocoamphoacetate, cocoamphodiacetate, lauroamphoacetate, lauroamphodiacetate, and mixtures thereof.

Zwitterionic detersive surfactants suitable for use in the hair care composition include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. In another embodiment, zwitterionics such as betaines are selected.

Non limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

### D. Carrier

The hair care compositions can be in the form of pourable liquids (under ambient conditions). Such compositions will therefore typically comprise a carrier, which is present at a level of from about 20 wt % to about 95 wt %, or even from about 60 wt % to about 85 wt %. The carrier may comprise water, or a miscible mixture of water and organic solvent, and in one aspect may comprise water with minimal or no significant concentrations of organic solvent, except as otherwise incidentally incorporated into the composition as minor ingredients of other essential or optional components.

The carrier useful in embodiments of the hair care compositions of the present invention includes water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

### E. Additional Components

The hair care compositions of the present invention may further comprise one or more additional components known for use in hair care or personal care products, provided that the additional components are physically and chemically compatible with the essential components described herein, or do not otherwise unduly impair product stability, aesthetics or performance. Such optional ingredients are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt % to about 10 wt % by weight of the personal care compositions.

Non-limiting examples of additional components for use in the hair care composition include conditioning agents (e.g., silicones, hydrocarbon oils, fatty esters), natural cationic deposition polymers, synthetic cationic deposition polymers, anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

1. Conditioning Agent

In one embodiment of the present invention, the hair care care compositions comprise one or more conditioning agents. Conditioning agents include materials that are used to give a particular conditioning benefit to hair and/or skin. The conditioning agents useful in the hair care compositions of the present invention typically comprise a water-insoluble, water-dispersible, non-volatile, liquid that forms emulsified, liquid particles. Suitable conditioning agents for use in the hair care composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix.

One or more conditioning agents are present from about 0.01 wt % to about 10 wt %, from about 0.1 wt % to about 8 wt %, and from about 0.2 wt % to about 4 wt %, by weight of the composition.

a. Silicones

The conditioning agent of the compositions of the present invention can be an insoluble silicone conditioning agent. The silicone conditioning agent particles may comprise volatile silicone, non-volatile silicone, or combinations thereof. In one embodiment the conditioning agent is a non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, by weight of the composition, from about 0.1% to about 8%, from about 0.1% to about 5%, and from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609, which descriptions are incorporated herein by reference. The silicone conditioning agents for use in the compositions of the present invention can have a viscosity, as measured at 25Â° C., from about 20 to about 2,000,000 centistokes (“csk”), from about 1,000 to about 1,800,000 csk, from about 50,000 to about 1,500,000 csk, and from about 100,000 to about 1,500,000 csk.

The dispersed silicone conditioning agent particles typically have a volume average particle diameter ranging from about 0.01 micrometer to about 50 micrometer. For small particle application to hair, the volume average particle diameters typically range from about 0.01 micrometer to about 4 micrometer, from about 0.01 micrometer to about 2 micrometer, from about 0.01 micrometer to about 0.5 micrometer. For larger particle application to hair, the volume average particle diameters typically range from about 5 micrometer to about 125 micrometer, from about 10 micrometer to about 90 micrometer, from about 15 micrometer to about 70 micrometer, from about 20 micrometer to about 50 micrometer.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

i. Silicone Oils

Silicone fluids include silicone oils, which are flowable silicone materials having a viscosity, as measured at 25° C., less than 1,000,000 csk, from about 5 csk to about 1,000,000 csk, from about 100 csk to about 600,000 csk. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula (I):

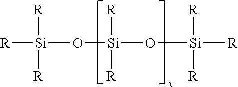

wherein R is aliphatic, in one embodiment alkyl or alkenyl, or aryl, R can be substituted or unsubstituted, and x is an integer from 1 to about 8,000. Suitable R groups for use in the compositions of the present invention include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Suitable alkyl and alkenyl substituents are C₁ to C₅ alkyls and alkenyls, from C₁ to C₄, alternatively from C₁ to C₂. The aliphatic portions of other alkyl-, alkenyl-, or alkynyl-containing groups (such as alkoxy, alkaryl, and alkamino) can be straight or branched chains, and can be from C₁ to C₅, from C₁ to C₄, from C₁ to C₃, from C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length can be as described herein.

ii. Amino and Cationic Silicones

Cationic silicone fluids suitable for use in the compositions of the present invention include, but are not limited to, those which conform to the general formula (II):

(R¹)ₐG₃₋ₐ-Si—(—OSiG₂)ₙ-(—OSiG_{b}(R¹)_{2-b})ₘ—O—SiG₃₋ₐ(R¹)ₐ

wherein G is hydrogen, phenyl, hydroxy, or C₁-C₈ alkyl, in one embodiment is methyl; a is 0 or an integer having a value from 1 to 3, in one embodiment 0; b is 0 or 1, in one embodiment 1; n is a number from 0 to 1,999, and in one embodiment from 49 to 499; m is an integer from 1 to 2,000, in one embodiment from 1 to 10; the sum of n and m is a number from 1 to 2,000, in one embodiment from 50 to 500; R¹ is a monovalent radical conforming to the general formula CqH_{2q}L, wherein q is an integer having a value from 2 to 8 and L is selected from the following groups:

—N(R²)CH₂—CH₂—N(R²)₂

—N(R²)₂

—N(R²)₃A⁻

—N(R²)CH₂—CH₂—NR²H₂A⁻

wherein R² is hydrogen, phenyl, benzyl, or a saturated hydrocarbon radical, in one embodiment an alkyl radical from about C₁ to about C₂₀, and A⁻ is a halide ion.

In one embodiment the cationic silicone corresponding to formula (II) is the polymer known as “trimethylsilylamodimethicone”, which is shown below in formula (III):

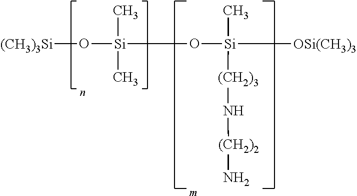

Other silicone cationic polymers which may be used in the compositions of the present invention are represented by the general formula (IV):

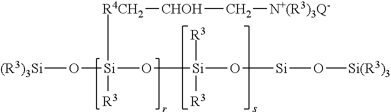

wherein R³ is a monovalent hydrocarbon radical from C₁ to C₁₈, in one embodiment an alkyl or alkenyl radical, such as methyl; R₄ is a hydrocarbon radical, in one embodiment a C₁ to C₁₈ alkylene radical or a C₁₀ to C₁₋₈ alkyleneoxy radical, in one embodiment a C₁ to C₈ alkyleneoxy radical; Q⁻ is a halide ion, in one embodiment chloride; r is an average statistical value from 2 to 20, in one embodiment from 2 to 8; s is an average statistical value from 20 to 200, in one embodiment from 20 to 50. One suitable example of a polymer in this class is known as UCARE SILICONE ALE 56®, available from Union Carbide.

iii. Silicone Gums

Other silicone fluids suitable for use in the compositions of the present invention are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, all of which are incorporated herein by reference. Specific non-limiting examples of silicone gums for use in the compositions of the present invention include polydimethylsiloxane, (polydimethylsiloxane)(methylvinylsiloxane)copolymer, poly(dimethylsiloxane)(diphenyl siloxane)(methylvinylsiloxane)copolymer and mixtures thereof

iv. High Refractive Index Silicones

Other non-volatile, insoluble silicone fluid conditioning agents that are suitable for use in the compositions of the present invention are those known as “high refractive index silicones,” having a refractive index of at least about 1.46, at least about 1.48, at least about 1.52, or at least about 1.55. The refractive index of the polysiloxane fluid will generally be less than about 1.70, typically less than about 1.60. In this context, polysiloxane “fluid” includes oils as well as gums. The high refractive index polysiloxane fluid includes those represented by general Formula (I) above, as well as cyclic polysiloxanes such as those represented by Formula (V) below:

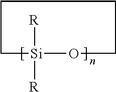

wherein R is as defined above, and n is a number from about 3 to about 7, or from about 3 to about 5.

The high refractive index polysiloxane fluids contain an amount of aryl-containing R substituents sufficient to increase the refractive index to the desired level, which is described herein. Additionally, R and n must be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, at least about 20%, at least about 25%, at least about 35%, at least about 50%. Typically, the degree of aryl substitution will be less than about 90%, more generally less than about 85%, and in one embodiment from about 55% to about 80%. Suitable high refractive index polysiloxane fluids have a combination of phenyl or phenyl derivative substituents, with alkyl substituents, in one embodiment C₁-C₄ alkyl (in one embodiment methyl), hydroxy, or C₁-C₄ alkylamino (especially—R⁴NHR⁵NH2 wherein each R⁴ and R⁵ independently is a C₁-C₃ alkyl, alkenyl, and/or alkoxy).

When high refractive index silicones are used in the compositions of the present invention, they can be used in solution with a spreading agent, such as a silicone resin or a surfactant, to reduce the surface tension by a sufficient amount to enhance spreading and thereby enhance the glossiness (subsequent to drying) of hair treated with the compositions.

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984), all of which are incorporated herein by reference.

v. Silicone Resins

Silicone resins may be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Suitable silicone resins for use in the compositions of the present invention include, but are not limited to MQ, MT, MTQ, MDT and MDTQ resins. Methyl is a suitable silicone substituent. Other suitable silicone resins include MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, can be from about 4:1 to about 400:1, from about 9:1 to about 200:1, from about 19:1 to about 100:1, particularly when the silicone fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of silicone conditioning agent in the composition.

b. Organic Conditioning Oils

The conditioning agent of the hair care compositions of the present invention may also comprise at least one organic conditioning oil, either alone or in combination with other conditioning agents, such as the silicones described above.

i. Hydrocarbon Oils

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils can be from about C₁₂ to about C₁₉. Branched chain hydrocarbon oils, including hydrocarbon polymers, typically will contain more than 19 carbon atoms.

ii. Polyolefins

Organic conditioning oils for use in the hair care compositions of the present invention can also include liquid polyolefins, including liquid poly-α-olefins and/or hydrogenated liquid poly-a-olefins. Polyolefins for use herein are prepared by polymerization of C₄ to about C₁₄ olefenic monomers, and in one embodiment from about C₆ to about C₁₂.

iii. Fatty Esters

Other suitable organic conditioning oils for use as the conditioning agent in the hair care compositions of the present invention include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

iv. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones.

v. Fatty Alcohols

Other suitable organic conditioning oils for use in the personal care compositions of the present invention include, but are not limited to, fatty alcohols having at least about 10 carbon atoms, about 10 to about 22 carbon atoms, and in one embodiment about 12 to about 16 carbon atoms.

vi. Alkyl Glucosides and Alkyl Glucoside Derivatives

Suitable organic conditioning oils for use in the personal care compositions of the present invention include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

c. Other Conditioning Agents

i. Quaternary Ammonium Compounds

Suitable quaternary ammonium compounds for use as conditioning agents in the personal care compositions of the present invention include, but are not limited to, hydrophilic quaternary ammonium compounds with a long chain substituent having a carbonyl moiety, like an amide moiety, or a phosphate ester moiety or a similar hydrophilic moiety.

Examples of useful hydrophilic quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

ii. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

iii. Cationic deposition polymers

The hair care composition of the present invention may further comprise a cationic deposition polymer. Any known natural or synthetic cationic deposition polymer can be used herein. Examples include those polymers disclosed in U.S. Pat. No. 6,649,155; U.S. Patent Application Publication Nos. 2008/0317698; 2008/0206355; and 2006/0099167, which are incorporated herein by reference in their entirety.

The cationic deposition polymer is included in the composition at a level from about 0.01 wt % to about 2 wt %, in one embodiment from about 1.5 wt % to about 1.9 wt %, in another embodiment from about 1.8 wt % to about 2.0 wt %, in view of providing the benefits of the present invention.

The cationic deposition polymer is a water soluble polymer with a charge density from about 0.5 milliequivalents per gram to about 12 milliequivalents per gram. The cationic deposition polymer used in the composition has a molecular weight of about 100,000 Daltons to about 5,000,000 Daltons. The cationic deposition polymer is a low charge density cationic polymer.

In one embodiment, the cationic deposition polymer is a synthetic cationic deposition polymer. A variety of synthetic cationic deposition polymers can be used including mono- and di-alkyl chain cationic surfactants. In one embodiment, mono-alkyl chain cationic surfactants are chosen including, for example, mono-alkyl quaternary ammonium salts and mono-alkyl amines. In another embodiment, di-alkyl chain cationic surfactants are used and include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In another embodiment, the cationic deposition polymer is a naturally derived cationic polymer. The term, “naturally derived cationic polymer” as used herein, refers to cationic deposition polymers which are obtained from natural sources. The natural sources may be polysaccharide polymers. Therefore, the naturally derived cationic polymer may be selected from the group comprising starches, guar, cellulose, Cassia, locust bean, Konjac, Tara, galactomannan, tapioca, and synthetic polymers. In a further embodiment, cationic deposition polymers are selected from Mirapol® 100S (Rhodia), Jaguar® C17, poyDADMAC, Tapioca starch (Akzo), Triquat™, and mixtures thereof

d. Anionic Emulsifiers

A variety of anionic emulsifiers can be used in the shampoo composition of the present invention as described below. The anionic emulsifiers include, by way of illustrating and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

In addition, anionic emulsifiers that have acrylate functionality may also be used in the instant shampoo compositions. Anionic emulsifiers useful herein include, but aren't limited to: poly(meth)acrylic acid; copolymers of (meth)acrylic acids and its (meth)acrylates with C1-22 alkyl, C1-C8 alkyl, butyl; copolymers of (meth)acrylic acids and (meth)acrylamide; Carboxyvinylpolymer; acrylate copolymers such as Acrylate/C10-30 alkyl acrylate crosspolymer, Acrylic acid/vinyl ester copolymer/Acrylates/Vinyl Isodecanoate crosspolymer, Acrylates/Palmeth-25 Acrylate copolymer, Acrylate/Steareth-20 Itaconate copolymer, and Acrylate/Celeth-20 Itaconate copolymer; Polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. Neutralizing agents may be included to neutralize the anionic emulsifiers herein. Non-limiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof. Commercially available anionic emulsifiers include, for example, Carbomer supplied from Noveon under the tradename Carbopol 981 and Carbopol 980; Acrylates/C10-30 Alkyl Acrylate Crosspolymer having tradenames Pemulen TR-1, Pemulen TR-2, Carbopol 1342, Carbopol 1382, and Carbopol ETD 2020, all available from Noveon; sodium carboxymethylcellulose supplied from Hercules as CMC series; and Acrylate copolymer having a tradename Capigel supplied from Seppic. In another embodiment, anionic emulsifiers are carboxymethylcelluloses.

e. Benefit Agents

The benefit agents comprise a material selected from the group consisting of anti-dandruff agents; perfumes; brighteners; enzymes; perfumes; sensates in one aspect a cooling agent; attractants, anti-bacterial agents; dyes; pigments; bleaches; and mixtures thereof.

In one aspect said benefit agent may comprise an anti-dandruff agent. Such anti-dandruff particulate should be physically and chemically compatible with the essential components of the composition, and should not otherwise unduly impair product stability, aesthetics or performance.

According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt % to about 5 wt %, or from about 0.1 wt % to about 3 wt %, or from about 0.1 wt % to about 2 wt %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, fcharcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt % to about 5 wt %, or from about 0.1 wt % to about 3 wt %, or from about 0.3 wt % to about 2 wt %. In an embodiment, the azole anti-microbial active is ketoconazole. In an embodiment, the sole anti-microbial active is ketoconazole.

Embodiments of the present invention may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt % to about 10 wt %, or from about 0.01 wt % to about 7 wt %, or from about 0.1 wt % to about 5 wt % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+} A^{m-}_{x/m}·nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺ Aⁿ⁻_{(1=3y)/n}·H₂O where the two metal ions (M²⁺) may be the same or different. If they are the same and represented by zinc, the formula simplifies to [Zn₁₊ₓ(OH)₂]^{2x+} 2×A⁻·nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

Embodiments of the present invention may also comprise fatty alcohol gel networks, which have been used for years in cosmetic creams and hair conditioners. These gel networks are formed by combining fatty alcohols and surfactants in the ratio of 1:1 to 40:1 (in one embodiment from 2:1 to 20:1, in another embodiment from 3:1 to 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

Thus according to an embodiment, the fatty alcohol is included in the fatty alcohol gel network at a level by weight of from about 0.05 wt % to about 14 wt %. For example, the fatty alcohol may be present in an amount ranging from about 1 wt % to about 10 wt %, and in another embodiment from about 6 wt % to about 8 wt %.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20, are suitable.

Gel network preparation: A vessel is charged with water and the water is heated to about 74° C. Cetyl alcohol, stearyl alcohol, and SLES surfactant are added to the heated water. After incorporation, the resulting mixture is passed through a heat exchanger where the mixture is cooled to about 35° C. Upon cooling, the fatty alcohols and surfactant crystallized to form a crystalline gel network. Table 4 provides the components and their respective amounts for the gel network composition.

TABLE 4
Gel network components

| Ingredient | Wt. % |
|---|---|
| Water | 78.27% |
| Cetyl Alcohol | 4.18% |
| Steary Alcohol | 7.52% |
| Sodium laureth-3 sulfate (28% Active) | 10.00% |
| 5-Chloro-2-methyl-4-isothiazolin-3-one, Kathon CG | 0.03% |

The hair care compositions of the present invention may be presented in typical hair care formulations. They may be in the form of solutions, dispersion, emulsions, powders, talcs, encapsulated, spheres, spongers, solid dosage forms, foams, and other delivery mechanisms. The compositions of the embodiments of the present invention may be hair tonics, leave-on hair products such as treatment, and styling products, rinse-off hair products such as shampoos, and treatment products; and any other form that may be applied to hair.

According to one embodiment, the hair care compositions may be provided in the form of a porous, dissolvable solid structure, such as those disclosed in U.S. Patent Application Publication Nos. 2009/0232873; and 2010/0179083, which are incorporated herein by reference in their entirety. Accordingly, the hair care compositions comprise a chelant, a buffer system comprising an organic acid, from about 23% to about 75% surfactant; from about 10% to about 50% water soluble polymer; and optionally, from about 1% to about 15% plasticizer; such that the hair care composition is in the form of a flexible porous dissolvable solid structure, wherein said structure has a Percent open cell content of from about 80% to about 100%.

According to another embodiment, a hair care composition in the form of a porous dissolvable solid structure, comprising: a chelant; a buffer system comprising an organic acid from about 23% to about 75% surfactant; wherein said surfactant has an average ethoxylate/alkyl ratio of from about 0.001 to about 0.45; from about 10% to about 50% water soluble polymer; and from about 1% to about 15% plasticizer; and wherein said article has a density of from about 0.03 g/cm³ to about 0.20 g/cm³.

According to another embodiment, a hair care composition in the form of a viscous liquid comprising: a chelant; a buffer system comprising an organic acid from 5-20% surfactant and a polycarboxylate rheology modifier; wherein the polycarboxylate is specifically chosen to be effective at the high electrolyte levels resulting from the incorporation of the key buffer system and chelant required for this invention. Non-limiting examples include acrylates/C10-C30 alkyl acrylate crosspolymers such as Carbopol EDT2020, 1342,1382, etc. from Lubrizol. Rheology benefits of these actives in our embodiments include stability, ease of dispensing, smoothness of spreading, etc.

The hair care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The hair care composition may be in a single phase or a single product, or the hair care composition may be in a separate phases or separate products. If two products are used, the products may be used together, at the same time or sequentially. Sequential use may occur in a short period of time, such as immediately after the use of one product, or it may occur over a period of hours or days.

### Test Methods

It is understood that the test methods that are disclosed in the Test Methods Section of the present application should be used to determine the respective values of the parameters of Applicants' invention as such invention is described and claimed herein. Unless specified otherwise, a direct pH measurement is performed using a standard hydrogen electrode of the composition at 25° C.

Inductively Coupled Plasma—Atomic Emission Spectroscopic measurement: The metal content of hair samples was determined by inductively coupled plasma atomic spectroscopy (ICP-AES) with an Optima 5300 DV Optical Emission Spectrometer (Perkin Elmer Life and Analytical Sciences, Shelton, Conn., USA). Samples of 100 mg of hair is digested overnight with 2 ml of high purity concentrated nitric acid (70% v/v Aristar Plus, BDH Chemicals, Poole, Dorset, UK). This mixture also contains 150 μL of 100 ppm Yttrium internal standard (Inorganic Ventures, Christianburg, Va., USA). Samples are then heated to a temperature within the range from about 70° C. to about 80° C. for one hour, cooled to room temperature and diluted to 15 mL with deionized water. Each hair sample is analyzed in triplicate.

Well water is tested by spiking the sample with 150 μL of 100 ppm Yttrium internal standard (Inorganic Ventures, Christianburg, Va., USA). Each well water sample is analyzed in triplicate.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following are representative of hair care compositions encompassed by embodiments of the present invention. Composition A is a simple surfactant shampoo composition. Compositions B and C are conditioning shampoo compositions.

### Example 1

| Ingredient | A | B | C |
|---|---|---|---|
| Sodium Laureth Sulfate | | 6 | 10 |
| (SLE3S) | | | |
| Sodium Lauryl Sulfate (SLS) | 1.5 | 6 | 1.5 |
| Sodium Laureth Sulfate | 10.5 | | |
| (SLE1S) | | | |
| Cocamidopropyl betaine | 1 | 1 | |
| (CapB) | | | |
| Coconut monoethanol amide | | 0.85 | |
| (CMEA) | | | |
| Gel Network (C16OH/ | | | 2 |
| C18OH) | | | |
| Dimethicone | | 1 | 1 |
| Ethylene glycol distearate | | 1.5 | 1.5 |
| (EGDS) | | | |
| Jaguar ® C500 | | 0.25 | 0.15 |
| Citric acid | 0, 2 | 0, 2 | 0-2 |
| Ethylene diamine disuccinic | 0, 0.1, 0.5 | 0, 0.1, 0.5 | 0, 0.1, 0.5 |
| acid (EDDS) | | | |
| pH | 4.5 | 4.5 | 4.5 |
| Water-USP Purified & Minors | Q.S. to 100 | Q.S. to 100 | Q.S. to 100 |

### Example 2

The following are representative of hair care compositions encompassed by embodiments of the present invention.

| Ingredient | D | E | F | G |
|---|---|---|---|---|
| Water-USP Purified & | Q.S. | Q.S. to 100 | Q.S. | Q.S. |
| Minors | to 100 | | to 100 | to 100 |
| Sodium Laureth Sulfate | 12 | 12 | 12 | 12 |
| (SLE1S) | | | | |
| Sodium Lauryl Sulfate (SLS) | 1.5 | 1.5 | 1.5 | 1.5 |
| Cocoamidoproply Betaine | 1.7 | 1.7 | 2 | 2 |
| (CapB) | | | | |
| Gel Network | 1.0 | 1.0 | 2.0 | 2.0 |
| Guar | 0.3 | 0.3 | 0.1 | 0.2 |
| Hydroxypropyltrimonium | | | | |
| Chloride¹ | | | | |
| Ethylene Glycol Distearate | 1.5 | 1.5 | 1.5 | 1.5 |
| Dimethicone/Dimethiconol | 1.0 | 1.0 | 0.5 | 0.5 |
| Citric Acid | 1.0 | 1.0 | 1.0 | 1.0 |
| Sodium Citrate Dihydrate | 1.0 | 1.0 | 1.0 | 1.0 |
| Acrylates/C10-C30 alkyl | — | 0.3 | — | 0.3 |
| acrylate crosspolymers | | | | |
| EDDS | 0.1 | 0.1 | 0.5 | 0.5 |
| Kathon | 0.0005 | 0.0005 | 0.0005 | 0.0005 |
| Sodium Benzoate | 0.25 | 0.25 | 0.25 | 0.25 |
| Disodium EDTA | 0.1274 | 0.1274 | 0.1274 | 0.1274 |
| Perfume | 0.7 | 0.7 | 0.7 | 0.7 |
| Sodium Chloride¹ | 0-3 | 0-3 | 0-3 | 0-3 |
| Sodium Xylene Sulfonate¹ | 0-3 | 0-3 | 0-3 | 0-3 |

¹Levels adjusted to reach desired viscosity

The following are representative of hair care compositions encompassed by embodiments of the present invention and are useful as comparative examples.

### Example 3

| Ingredient | H | I | J | K | |
|---|---|---|---|---|---|
| Sodium Lauryl Sulfate (SLS) | 1.5 | 1.5 | 1.5 | 1.5 | Testing |
| Sodium Laureth Sulfate | 10.5 | 10.5 | 10.5 | 10.5 | Protocol: |
| (SLE1S) | | | | | |
| Cocamidopropyl betaine | 1 | 1 | 1 | 1 | |
| (CapB) | | | | | |
| Citric acid/citrate buffer | 0.4 | 0.4 | 0.4 | 2 | |
| Ethylene diamine disuccinic acid | 0 | 0.1 | 0.1 | 0.1 | |
| (EDDS) | | | | | |
| pH | 6.00 | 6.00 | 4.25 | 4.25 | |

Single source consumer hair is used for testing with a significant base-line number of calcium carbonate deposits in the cuticle. The hair is made into 1 g, 6 inch switches. These switches are divided into 4 treatment legs (2 per composition):

Composition H: (Control): Surfactant shampoo control at pH 6.00.
Composition I: Surfactant shampoo+0.1% EDDS at pH 6.00.
Composition J: Surfactant shampoo+0.1% EDDS at pH 4.25.
Composition K: Surfactant shampoo+0.1% EDDS+2% citric acid/citrate buffer at pH 4.25.

The water used for the testing is well water with 0.06 ppm copper and 291-325 ppm calcium. After 5, 10, 15 and 20 wash cycles with the test Compositions H-K, the hair is sampled for metals (Ca, Cu) using ICP-MS. Tables 5a and 5b below show the data from the ICP-MS metals analysis for samples taken from the hair switches after washing with the Compositions H-K for the listed number of cycles.

TABLE 5a
Copper (ppm) Data

| Composition # | Cycle 5 | Cycle 10 | Cycle 15 | Cycle 20 |
|---|---|---|---|---|
| H: Control pH 6.0 | 38.3 | 41.7 | 48.7 | 46.5 |
| I: +EDDS pH 6.0 | 28.1 | 29.0 | 32.9 | 31.9 |
| J: +EDDS pH 4.25 | 26.4 | 27.8 | 29.4 | 28.1 |
| K: +EDDS pH 4.25 | 24.3 | 25.3 | 26.4 | 25.7 |

TABLE 5b
Calcium (ppm) Data

| Composition # | Cycle 5 | Cycle 10 | Cycle 15 | Cycle 20 |
|---|---|---|---|---|
| H: Control pH 6.0 | 4164 | 4102 | 4739 | 4526 |
| I: +EDDS pH 6.0 | 3711 | 3931 | 4488 | 4385 |
| J: +EDDS pH 4.25 | 3705 | 3914 | 4219 | 3913 |
| K: +EDDS pH 4.25 | 3402 | 3517 | 3419 | 3149 |

A significant decrease in copper levels is seen for all the EDDS legs versus the control (Composition H) with no EDDS. The pH significantly lowers the copper removal (Composition J & K versus Composition I at cycle 20). The combination technology also shows significant calcium removal (Composition K versus Compositions I, J, and K at cycle 20).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a. from about 0.01 wt % to about 10 wt % of ethylene diamine disuccinic acid (EDDS) or salts thereof;
b. a buffer system comprising an organic acid and/or a salt thereof, wherein the organic acid is selected from an alpha-hydroxy acid or a polycarboxylic acid;
c. from about 2 wt % to about 50 wt % of a detersive surfactant; and
d. a carrier,
wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).

2. The hair care composition of claim 1, wherein the alpha-hydroxy acid selected from citric acid, malic acid, tartaric acid, or combinations thereof, or wherein the polycarboxylic acid is malonic acid.

3. The hair care composition of claim 1, wherein the organic acid has a molecular weight from about 90 g/mol to about 200 g/mol.

4. The hair care composition of claim 1, wherein the organic acid has log of stability constant for the organic acid with Ca⁺² equal to or greater than about 1.5.

5. The hair care composition of claim 1, wherein the organic acid has log of a conditional stability constant for the organic acid with Ca⁺² calculated at pH 5 equal to or greater than 1.5.

6. The hair care composition of claim 1, wherein the organic acid is citric acid.

7. The hair care composition of claim 1, wherein the detersive surfactant is selected from an anionic surfactant, cationic surfactant, non-ionic surfactant, amphoteric surfactants, or mixtures thereof.

8. The hair care composition of claim 1, wherein the detersive surfactant is present in an amount ranging from about 5 wt % to about 25 wt %.

9. The hair care composition of claim 1, further comprising a gel network comprising a fatty alcohol and a surfactant.

10. The hair care composition of claim 1, further comprising a polycarboxylate rheology modifier.

11. A hair care composition comprising:
a. from about 0.01 wt % to about 10 wt % of a chelant (L), wherein the chelant has
i. a log K_{CaL} of less than about −2, and
ii. a log K_{CuL} of greater than about 3 or a log K_{FeL} of greater than about 10, wherein the log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5;
b. a buffer system comprising an organic acid and/or a salt thereof, wherein the organic acid is selected from an alpha-hydroxy acid or a polycarboxylic acid;
c. from about 2 wt % to about 50 wt % of a detersive surfactant; and
d. a carrier,
wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).

12. The hair care composition of claim 11, wherein the alpha-hydroxy acid selected from citric acid, malic acid, tartaric acid, or combinations thereof, or wherein the polycarboxylic acid is malonic acid.

13. The hair care composition of claim 11, wherein the organic acid is citric acid.

14. The hair care composition of claim 11, wherein the organic acid has a molecular weight from about 90 g/mol to about 200 g/mol.

15. The hair care composition of claim 11, wherein the organic acid has log Stability Constant for the organic acid with Ca⁺² equal to or greater than about 1.5.

16. The hair care composition of claim 11, wherein the organic acid has log Conditional Stability Constant for the organic acid with Ca⁺² calculated at pH 5 equal to or greater than 1.5.

17. The hair care composition of claim 11, wherein the organic acid and calcium form a calcium chelate complex having an aqueous solubility at 25° C. of equal to or greater than the solubility of calcium carbonate.

18. The hair care composition of claim 11, wherein the chelant is selected from diethylenetriamine penta(methylene phosphonic acid) (DTPMP); ethylenediamine-N,N′-diglutaric acid (EDDG); ethylenediamine-N,N′-bis(2-hydroxyphenylacetic acid) (EDDHA); ethylene diamine disuccinic acid (EDDS); glutamic acid diacetic acid (GLDA); hexadentate aminocarboxylate (HBED); 2-hydroxypropylendiamin-N-N′-disucinnic acid (HPDDS); methylglycinediacetic acid (MGDA); salts thereof, derivatives thereof, or mixtures thereof.

19. The hair care composition of claim 11, wherein the chelant is ethylene diamine disuccinic acid (EDDS) or salts thereof.

20. The hair care composition of claim 11, wherein the detersive surfactant is selected from an anionic surfactant, cationic surfactant, non-ionic surfactant, amphoteric surfactants, or mixtures thereof.

21. The hair care composition of claim 11, wherein the hair care composition has a pH in a range from about 3 to about 6 at 25° C.

22. The hair care composition of claim 11, wherein the hair care composition has a pH in a range from about 4 to about 5 at 25° C.

23. The hair care composition of claim 11, wherein a 1:100 diluted sample of the hair care composition has a pH greater than about 3 and less than about 6, the diluted sample prepared from 1 part hair care composition and 100 parts water (v/v).

24. A method of reducing deposited mineral content on keratinous tissue, comprising:
1) contacting keratinous tissue with a hair care composition comprising:
a. from about 0.01 wt % to about 10 wt % of a chelant (L), wherein the chelant has
i. a log K_{CaL} of less than about −2, and
ii. a log K_{CuL} of greater than about 3 or a log K_{FeL} of greater than about 10, wherein the log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5;
b. a buffer system comprising an organic acid and/or a salt thereof, wherein the organic acid is selected from an alpha-hydroxy acid or a polycarboxylic acid;
c. from about 2 wt % to about 50 wt % of a detersive surfactant; and
d. a carrier,
wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v); and
2) rinsing the hair care composition from the keratinous tissue.

25. The method of claim 24, wherein a 1:100 diluted sample of the hair care composition has a pH greater than about 3 and less than about 6, the diluted sample prepared from 1 part hair care composition and 100 parts water (v/v).

26. A hair care composition in the form of a porous dissolvable solid structure, comprising:
a. from about 0.01 wt % to about 10 wt % of a chelant (L), wherein the chelant has
i. a log K_{CaL} of less than about −2, and
ii. a log K_{CuL} of greater than about 3 or a log K_{FeL} of greater than about 10, wherein the log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5;
b. a buffer system comprising an organic acid and/or a salt thereof, wherein the organic acid is selected from an alpha-hydroxy acid or a polycarboxylic acid, wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v).
c. from about 23 wt % to about 75 wt % of a detersive surfactant;
d. from about 10 wt % to about 50 wt % of a water soluble polymer; and optionally, from about 1 wt % to about 15 wt % of a plasticizer; such that the hair care composition is in the form of the flexible porous dissolvable solid structure, wherein said structure has a Percent open cell content of from about 80 wt % to about 100 wt %.

27. A hair care composition in the form of a porous dissolvable solid structure, comprising:
a. from about 0.01 wt % to about 10 wt % of a chelant (L), wherein the chelant has
i. a log K_{CaL} of less than about −2, and
ii. a log K_{CuL} of greater than about 3 or a log K_{FeL} of greater than about 10, wherein the log K_{CaL} is the log of a conditional stability constant of the chelant with Ca⁺² calculated at pH 5, the log K_{CuL} is the log of a conditional stability constant of the chelant with Cu⁺² calculated at pH 5, and the log K_{FeL} is the log of a conditional stability constant of the chelant with Fe⁺³ calculated at pH 5;
b. a buffer system comprising an organic acid and/or a salt thereof, wherein the organic acid is selected from an alpha-hydroxy acid or a polycarboxylic acid, wherein the buffer system is present in a sufficient quantity to provide the hair care composition with a pH from about 2 to about 6 at 25° C., and wherein a 1:10 diluted sample of the hair care composition has a pH greater than about 3 and less than about 6, the diluted sample prepared from 1 part hair care composition and 10 parts water (v/v);
c. from about 23 wt % to about 75 wt % of a detersive surfactant; wherein said detersive surfactant has an average ethoxylate/alkyl ratio of from about 0.001 to about 0.45;
d. from about 10 wt % to about 50 wt % of a water soluble polymer; and
e. from about 1 wt % to about 15 wt % of a plasticizer; and wherein said structure has a density of from about 0.03 g/cm³ to about 0.20 g/cm³.
